(19) United States
(12) Patent Application Publication
(10) Pub. No.: US 2026/0110637 A1
(43) Pub. Date: Apr. 23, 2026

(54) COMBINED SENSOR DETECTION PLATFORM

(71) Applicant: Purdue Research Foundation, West Lafayette, IN (US)

(72) Inventors: J. Paul Robinson, West Lafayette, IN (US); Bartlomiej P. Rajwa, West Lafayette, IN (US); Euiwon Bae, West Lafayette, IN (US); Sungho Shin, West Lafayette, IN (US)

(21) Appl. No.: 19/116,091

(22) PCT Filed: Oct. 3, 2023
(86) PCT No.: PCT/US2023/034336

§ 371 Date: Mar. 27, 2025

Related U.S. Application Data
(60) Provisional application No. 63/412,702, filed on Oct. 3, 2022.

(51) Int. Cl. G01N 21/71; G01J 3/02; G01J 3/443; G01N 21/65; G01N 21/94; G01N 33/02

(52) U.S. Cl. CPC G01N 21/718; G01J 3/0208; G01J 3/0218; G01J 3/0227; G01J 3/0272; G01J 3/443; G01N 21/65; G01N 21/94; G01N 33/02; G01N 2201/0221

(57) ABSTRACT

The invention generally relates to systems and methods for combined spectroscopy systems. In certain embodiments, laser-induced breakdown spectroscopy (LIBS) and Raman spectroscopy can be combined in a single, handheld device using a single spectrometer and two sources for simultaneous analysis using both methods.

### RELATED APPLICATION

The present application claims the benefit of and priority to U.S. provisional patent application No. 63/412,702, filed Oct. 3, 2022, the content of which is incorporated by reference herein in its entirety.

### GOVERNMENT SUPPORT

This invention was made with government support under Agreement Nos. 59-8072-1-002 and 58-8042-0-061 awarded by the United States Department of Agriculture. The government has certain rights in the invention.

### FIELD OF THE INVENTION

The invention generally relates to methods and devices for combined, simultaneous analysis by Raman and laser-induced breakdown spectroscopy (LIBS).

### BACKGROUND

Food should be acceptable and safe levels of adulterants, contaminants or any other substances that may make food hazardous to human health. Food safety is a growing problem because of potential food adulteration and contamination that can be a source of infection or toxic poisoning. Specifically, the increasing abundance of microplastics and heavy metals is a major contributor to soil, water, and food contamination. Heavy metals have also been reported to accumulate on microplastic particles, resulting in potentially harmful effects. Agricultural products can be chemically and physically contaminated at different points of the food supply chain, including farming/production, packing, shipping, storage, and distribution stages. In addition, food adulteration has been another critical concern since the beginning of civilization, as it not only decreases the quality of food products but also results in several ill effects on health. Advanced development of innovative technologies and systematic approaches is necessary for foodborne hazard detection and characterization for improving food safety.

Various technologies are currently available for the analysis of food concerns such as vibrational spectroscopies, and mass spectrometry techniques. Among conventional techniques, laser-induced breakdown spectroscopy (LIBS) and Raman system are emerging as a promising technology due to their capability for real-time multiplex and in-air analysis. LIBS is a technique to measure the spectral intensity of a plasma generated by an intense-pulsed laser for the qualitative or quantitative elemental analysis of the target. LIBS is the only technology capable of real-time analysis of all kinds of elemental components with relatively less restrictions. Raman is also a spectroscopic technique to measure the spectral intensity of Raman scattering signals from a laser by providing a structural fingerprint from which molecules can be identified. Raman also allows for real-time analysis but is more time consuming than LIBS analysis. However, Raman has the advantage of providing molecular analysis while remaining non-destructive to the target. Because of these advantages, handheld devices for both LIBS and Raman approaches have been developed and are commercially available.

Combined LIBS and Raman systems have been developed for the purpose of providing both elemental and molecular analysis on targets since the two methods use standoff detection. However, there are significant challenges in conventional combined LIBS and Raman systems. First, there are size limitations due to the complexity of the design complicating handheld use. One of those size considerations is that most conventional combination LIBS and Raman devices are operated with an intensified charged coupled detector (ICCD) based spectrometer resulting in a large instrument. In addition, one or two lasers and two spectrometers to detect each LIBS and Raman signal require significant space inside these system designs. Furthermore, studies in food sciences have suggested that the existing designs are not optimized for food products with the primary target being mineral analysis to this point.

### SUMMARY

Systems and methods of the invention may include a handheld LIBS and Raman combined system. Methods may include the use of such a device for the detection and/or characterization of biological, chemical, and physical contaminants. As noted above, such contaminants are high value concerns in the food industry because simultaneous elemental and molecular analysis that can be implemented. In certain embodiments, devices may integrate a visible laser for Raman and may use of a single spectrometer for both LIBS and Raman signal detection. Devices of the invention have the potential for in-field food analysis due to the reduced complexity allowing for a handheld size and enhanced simultaneous detection.

Combined LIBS and Raman using a visible laser as described herein addresses the aforementioned problems and provides a capability for a handheld-sized device and higher spatial and sensitivity peaks on food targets. Devices and methods of the invention can provide simultaneous LIBS and Raman detection in a handheld-size device for the purpose of food adulteration and contaminants detection through real-time elemental and molecular analysis of the target. Such applications can replace traditional time-consuming inspections. Advantages afforded by systems and methods of the invention may include handheld size, light weight, low power draw, battery operation, and LIBS & Raman analysis. Systems may use the same spectrometer for detection of both signals providing a more cost-effective device with a single spectrometer and single collection optics. LIBS and Raman detection may be performed sequentially or simultaneously. The data fusion of both methods can enhance classification accuracy, especially in food adulteration analysis. The combination of elemental and molecular analysis is particularly useful in food contamination detection based on the high sensitivity peak for Raman (low absorption on food and water) and the high spatial resolution for both LIBS and Raman (tight focusing).

Systems and methods of the invention provide a technology that can utilize a combination of LIBS and Raman in a handheld device for the detection and subsequent classification of a variety of molecular or elemental species. The simultaneous detection approach advantageously allows for classification of molecular species that are difficult or impossible using either detection modality individually.

In certain aspects systems of the invention may include a first excitation source applying a first excitation energy to a point on a sample stage; a second excitation source applying a second excitation energy to the point simultaneously to the first excitation energy; and a single spectrometer positioned to receive and independently process energy emitted from the point in response to both the first and second excitation energies.

The first source may be a pulsed excitation source. The first source may be a laser-induced breakdown spectroscopy (LIBS) source. The first source may be a near-infrared region wavelength laser. In some embodiments, the first source can be a laser having a wavelength of about 1064 nm, energy of about 10 mJ, and about a 0.2 nm FWHM beam width, and applied in a pulse of about 6 ns. The second source may be a continuous excitation source. The second source can be a Raman source. The second source may be a visible laser. In some embodiments, the second source may be a laser having a wavelength of about 532 nm, energy of about 5 mW, and about a 0.3 nm FWHM beam width.

Systems may further comprise one or more mirrors and one or more lenses to direct and focus the first excitation energy and the second excitation energy on the point on the sample stage. The one or more mirrors may comprise a dichroic mirror operable to reflect excitation energy from the second excitation source and allow excitation energy from the first excitation source to pass therethrough. The one or more lenses can comprise a focusing lens.

In certain embodiments systems may comprise collection optics associated with the single spectrometer and one or more collecting lenses positioned between the point and the collection optics. Systems may further comprise a notch filter positioned between two collecting lenses between the point and the collection optics. The notch filter can be tuned to filter out a wavelength of the second source.

The collection optics may comprise an optical fiber coupled to the single spectrometer. The single spectrometer may be a visible range spectrometer. The system may be contained in a container having a total volume of about 750 cm³ or less. The system may be contained in a container having a longest linear dimension of about 15 cm. In some embodiments, the first source may be a pulsed excitation source and die second source is a continuous excitation source, the system further comprising a processor coupled to a tangible non-transient memory operable to acquire data from the single spectrometer for the second source in between pulsing of die first source. The sample stage can be a moveable in three axes.

Aspects of the invention may include methods for sample analysis such as applying a first excitation energy from a first excitation source to a point on a sample stage; simultaneously applying a second excitation energy from a second excitation source to the point to the first excitation energy; receiving energy emitted from the point in response to both the first and second excitation energies at a single spectrometer; and independently processing signals received in response to the first and second excitation energies. Methods may include directing and focusing the first excitation energy and the second excitation energy on the point on the sample stage using one or more mirrors and one or more lenses.

In some embodiments, the one or more mirrors may comprise a dichroic mirror, with the method further comprising using the dichroic mirror to reflect excitation energy from the second excitation source while allowing excitation energy from the first excitation source to pass therethrough. Methods may further comprise collecting emissions from the point using collection optics associated with the single spectrometer and one or more collecting lenses positioned between the point and the collection optics. Methods may include filtering emissions from the point with a notch filter positioned between two collecting lenses between the point and the collection optics.

### BRIEF DESCRIPTION OF THE DRAWINGS

FIG. 1 shows an exemplary portable combined LIBS/Raman system using a single visible light spectrometer.

FIG. 2 shows an exemplary combined LIBS/Raman system using a visible light spectrometer for LIBS detection and a near-infrared range (NTR) spectrometer for Raman detection.

FIG. 3A shows a 2D description of a handheld device operated by a battery and integrated electronics FIG. 3B shows a 3D design of detailed measurement body as shown in FIG. 3A. It contains optical structures, miniature stage, and two lasers which are constructed using conventional structures such as cage optics.

FIG. 4 shows validation tests of five different reference materials; 1. PS, 2. PE, 3. PP, 4. Boron, 5. Sulfur. (A) 1064 nm Raman data, (B) 532 nm Raman data, and (C) Reference device (635 nm laser with ICCD).

FIG. 5 shows a timeline of sequential data acquisition of LIBS or Raman.

FIG. 6 shows a timeline of simultaneous data acquisition of LIBS and Raman.

FIG. 7 shows a diagram of data processing for both LIBS and Raman.

FIG. 8 shows PE powder screening on cheese surface.

FIG. 9 shows PMMA powder screening on cheese surface.

FIG. 10 shows PS powder screening on cheese surface.

FIG. 11 shows total normalization of 50 averaged LIBS signals in cheese surface and polymer bead.

FIG. 12 shows boron powder detection in cheese surface.

FIG. 13 shows zinc powder detection in cheese surface.

FIG. 14 shows chromium powder detection in cheese surface.

FIG. 15 shows glyphosate Raman data for (A) an exemplary device of the invention (50 accumulated spectra taking 25 sec), and (B) reference device (one single shot data acquired from ICCD taking 1 sec).

FIG. 16 shows 5 averaged LIBS spectra of Glyphosate after total normalization for (A) full spectral range, and (B) specified spectral range.

FIG. 17 shows measured data for three different cheeses using (A) LIBS (50 averaged spectra after total normalization), and (B) Raman (50 averaged spectra after background removal).

FIG. 18 shows classification of three different cheeses for (A) Raman data only, (B) LIBS data only, (C) combined LIBS and Raman, and (D) combined LIBS and Raman with auto feature selection.

FIG. 19 shows measured data for five different coffee beans for (A) LIBS (50 averaged spectra after total normalization), and (B) Raman (50 averaged spectra after background removal).

FIG. 20 shows classification of five different coffee beans for (A) Raman data only, (B) LIBS data only, (C) combined LIBS and Raman, and (D) combined LIBS and Raman with auto feature selection.

FIG. 21 shows measured data for three different polymer beads for (A) LIBS (50 averaged spectra after total normalization), and (B) Raman (50 averaged spectra after background removal).

FIG. 22 shows classification of three different polymer beads for (A) Raman data only, (B) LIBS data only, (C) combined LIBS and Raman, and (D) combined LIBS and Raman with auto feature selection.

FIG. 23 shows classification summary plots for (A) Cheeses, (B) Coffee beans, and (C) Polymer beads with 10 running circles.

FIG. 24 shows a front view (FIG. 24A) and a side view (FIG. 24B) of an exemplary device with 532 nm Raman and LIBS detection.

FIG. 25 shows a side view of a measurement body according to certain embodiments.

FIG. 26 shows other views of the device in FIG. 24 with two lasers and two spectrometers corresponding to the two collection modalities.

FIG. 27 shows another embodiment of a handheld device.

FIG. 28 shows components of an exemplary handheld device

### DETAILED DESCRIPTION

The invention generally relates to the combination of two spectroscopic techniques such as laser-induced breakdown spectroscopy (LIBS) and Raman spectroscopy in a single device. The device may be portable and capable of simultaneously collecting both LIBS and Raman signals.

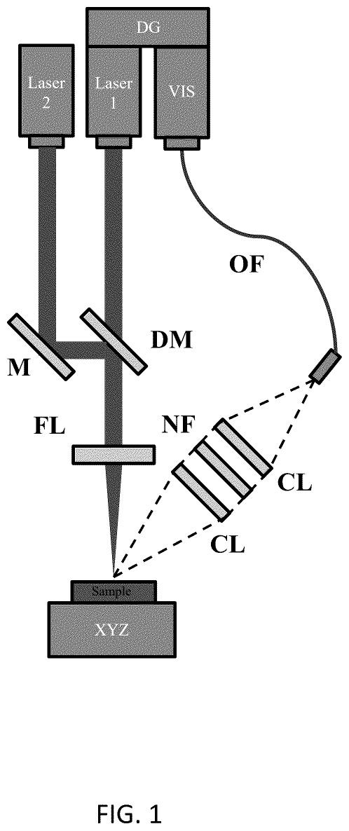

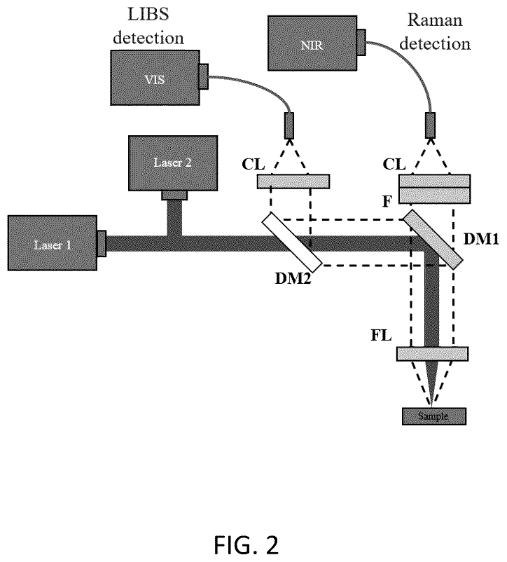

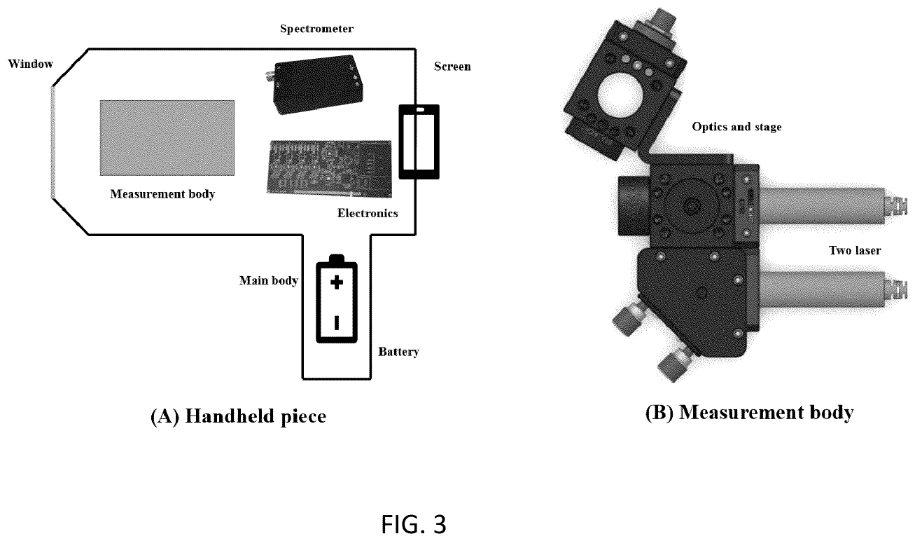

An exemplary device is shown in FIG. 1 having a combined LIBS (Laser 1, 1064 nm, pulse laser) and Raman (Laser 2, 532 nm, continuous laser) system in a single spectrometer. In particular, tight focus of two different lasers can be aligned by a dichroic mirror (DM) and a focusing lens (FL). Simultaneous LIBS and Raman signals can be collected through two collection lenses (CL) with a notch filter (NF) via an optical fiber (OF). In certain embodiments, the specifications of the various components shown in FIG. 1 may include Laser 1:1064 nm wavelength, 10 mJ (6 ns pulse, 0.2 nm FWHM); Laser 2:532 nm wavelength, 5 mW (0.3 nm FWHM); VIS:350-600 spectral range, 25 μm slit, 200-2300 cm⁻¹ Raman range; DG:Delay generator; M:Reflection mirror; DM:Dichroic mirror, @650 nm T; FL:Focusing lens, f=50 mm; CL:Collection lens, f=50 mm; NF:Notch filter, @532 nm block; OF:optical fiber, 1 mm core diameter; and XYZ:Three-axis manual stage. FIGS. 1 and 2 show two different schematics of combined LIBS and Raman systems. Two different excitation sources for Raman (1064 nm in FIG. 2), 532 nm in FIG. 1) are used for hardware optimization. Combined LIBS and Raman systems contain a pulsed laser for LIBS signal, a continuous wave (CW) laser for Raman, spectrometer, and optical structure. The miniature design as shown in FIG. 1 uses a single spectrometer where M is mirror, DM is dichroic mirror, FL is focusing lens, CL is collection lens, NF is notch filter, OF is optical fiber. The system dimension can be reduced to as little as 15×10×5 cm³ for handheld applications, however, in some embodiments, an additional spectrometer and optics may be required when an NIR wavelength is used as a Raman source since it needs at least one spectrometer and collection optics for both as depicted in FIG. 2. Both sequential or simultaneous LIBS and Raman signals can be detected in a single compact spectrometer. In detail, a few nano seconds of pulsed laser can immediately create a plasma emission signal while Raman scattering is continuously generated by the CW laser. Specific dichroic mirror allows NIR laser energy to pass through and reflects VIS laser to the target. All signals are collected through collection optics into a spectrometer including VIS range LIBS spectra and about 2000 cm⁻¹ range Raman spectra. The notch filter blocks the CW laser to prohibit direct reflection from CW laser source. FIG. 3 shows a 3D design of (A) a handheld piece, which contains window cavity, measurement body, spectrometer, electronics, battery, and screen and (B) a measurement main body, which contains optical structure, miniature stage, and two lasers.

The disclosed system provides the advantages of a compact design with the possibility of a handheld device. Furthermore, systems and methods herein allow for simultaneous detection of LIBS and Raman spectra, which can handle both elemental and molecular information from target. Furthermore, as discussed below, such a system can be optimized for food analysis. The combination of NIR pulsed laser and 532 nm CW laser may affect relatively high sensitivity on food analysis as well as real-time in-field analysis due to handheld design.

An exemplary device using two spectrometers (one visible light and one NIR) is shown in FIG. 2. FIG. 2 shows a device consisting of two NIR lasers for LIBS and Raman. This design requires two different spectrometers having a visible range for LIBS, and near-infrared range for Raman detection where VIS:VIS spectrometer for LIBS; NIR:NIR spectrometer for Raman; FL:focusing lens; DM1:dichroic mirror (R: ↓1100 nm); DM2:dichroic mirror (R: ↓900 nm); CL:collection lens; and F:filter.

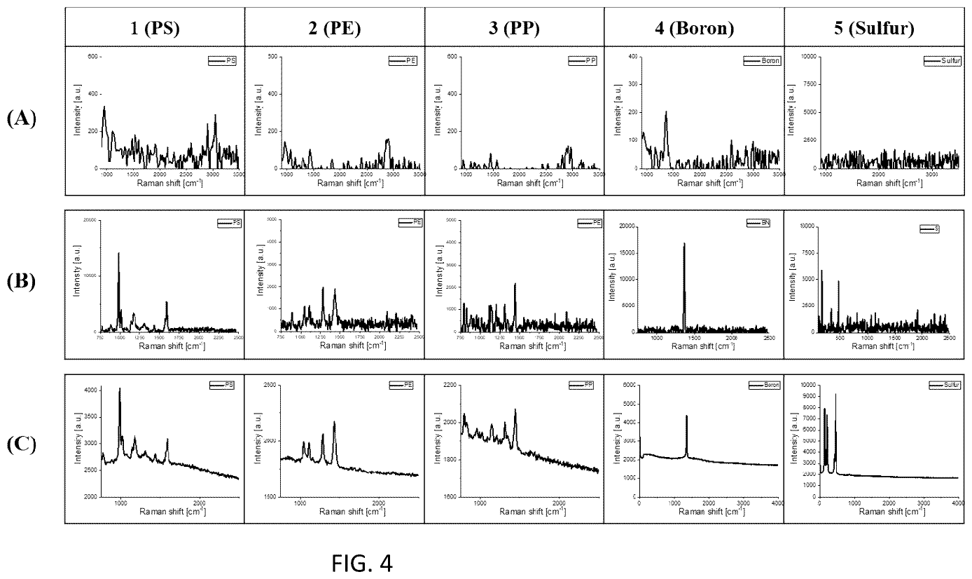

FIG. 4 shows a validation Raman test using five different reference materials (All samples are described in detail below). 10 accumulated spectra in FIG. 4A was measured by NIR source as shown in FIG. 2. Instead, 50 accumulated spectra in FIG. 4B was measured by 532 nm wavelength as shown in FIG. 1. The reference spectra of single spectra (FIG. 4C) was measured from a benchtop system, which consists of 635 nm CW laser and ICCD spectrometer installed in microscope. Background subtraction and filtering were conducted in all measured spectra while raw single spectra was presented in a refence spectra (FIG. 4C). Although different compact spectrometers were compared, it was shown that Raman signal from 532 nm wavelength had better sensitivity than that of 1064 nm wavelength. For that reason, NIR Raman detection may benefit from a more sensitive sensor and a cooling option resulting in additional bulk and expense.

In addition, there are three more reasons to choose a 532 nm wavelength source. First, the device needs only one single spectrometer since about 350-650 nm spectral range is required for LIBS and 550-650 spectral range for Raman. This selection VIS range spectrometer has high sensitivity and cost-effective benefits for compact design. Second, 532 nm wavelength has benefits to avoid photodegradation due to lower absorption in food materials inducing relatively higher Raman signal. Similarly, 532 nm has been shown to provide a higher enhancement than 633 nm and 780 nm wavelengths while detecting pesticide (Thiram) spreading on an apple surface. Third, tighter focusing could be achieved than with another common wavelength (785 nm). Theoretically, the radius of focus spot is linear on wavelength value in Gaussian beam optics. For example, Raman research to detect a nano particle under 1 μm size has been performed using a 53 nm excitation source. These devices are normally included with confocal microscope or SEM instrument.

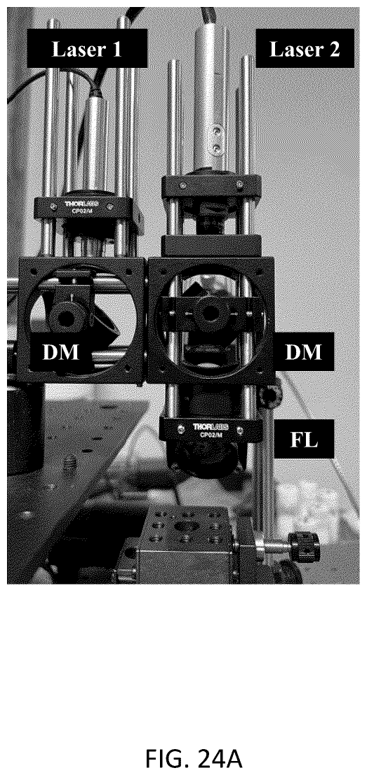

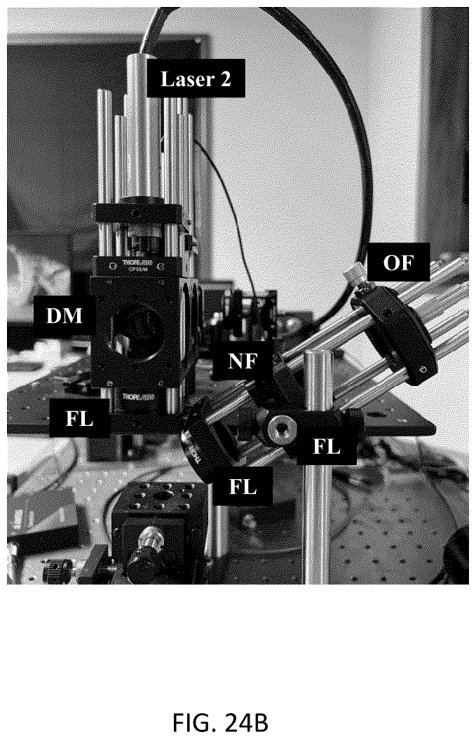

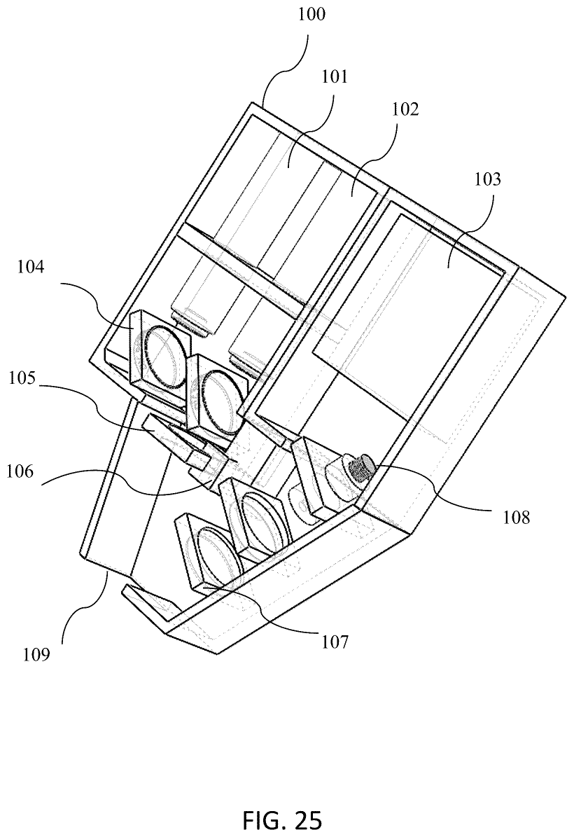

FIG. 24 shows a front view (FIG. 24A) and a side view (FIG. 24B) of an exemplary device with 532 nm Raman and LIBS detection. The dimensions of the device are about 20 cm by 10 cm by 15 cm which can be further reduced by employing a half lens and structure as shown in FIG. 25. FIG. 25 shows a side view of a measurement body according to certain embodiments where 100:measurement body; 101:CW laser; 102:pulsed laser; 103:VIS spectrometer; 104:mirror bundle; 105:focusing lens; 106:miniature three-axis stage; 107 collection optics; 108:optical fiber; and 109:window cavity.

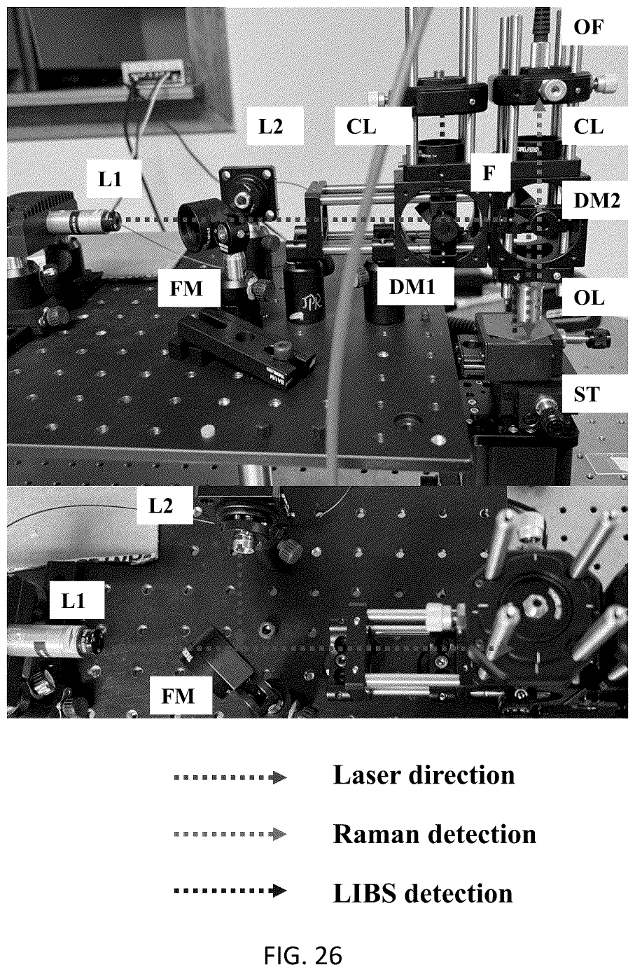

FIG. 26 shows other views of the device in FIG. 24 with two lasers and two spectrometers corresponding to each collection modality. L1:pulsed laser @1064 nm (0-10 mJ); L2:CW laser @1064 nm (0-500 mW); FM:flip mirror; DM1:dichroic mirror (@900 nm↑T); DM2:dichroic mirror (@1100 nm↑T); OL:objective lens×10 (f 16.5 mm); F:filter (@1100 nm↑T); CL:collection lens (f 50 mm); T:XYZ stage.

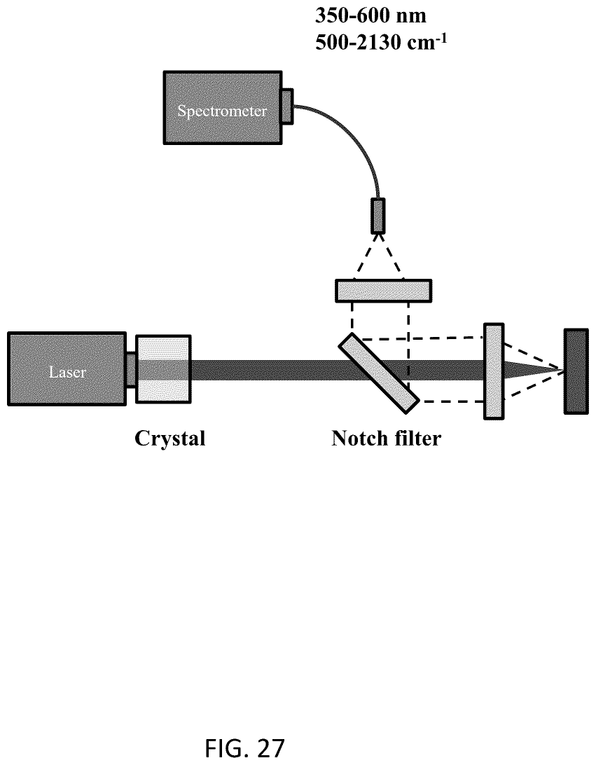

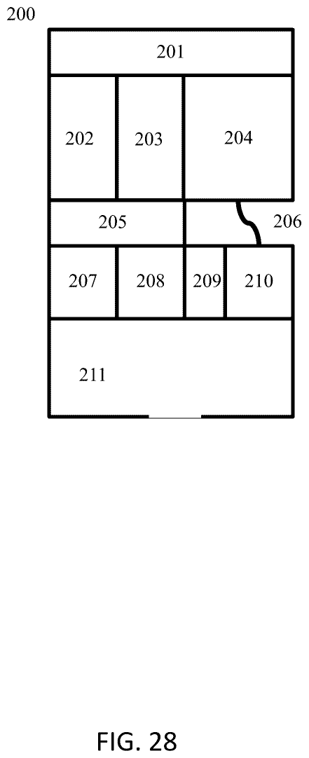

FIG. 27 shows another embodiment of a handheld device. FIG. 28 shows components of an exemplary handheld device with 200:main body; 201:power and control panel; 202:CW laser; 203:pulsed laser; 204:VIS spectrometer; 205:mirror bundle; 206:optical fiber; 207: buffer gas inlet; 208:focusing lens; 209:miniature three-axis stage; 210:collection optics; and 211:sample cage.

Exemplary excitation energy sources and spectrometer detectors can include the following:

LIBS laser (e.g., MicroJewel available from
Quantum Composers, Bozeman Montana)

| | | |
|---|---|---|
| Wavelength | 1064 | nm |
| Pulse energy | 10 | mJ |
| Pulse duration | 6 | ns |

| | |
|---|---|
| Spectral width | 0.2 nm FWHM |

LIBS detection (e.g., Avaspec-mini available
from Avantes, Apeldoorn, The Netherlands)

| Spectral range | 350-600 | nm |
|---|---|---|
| Spectral resolution | 0.3 | nm |
| Slit size | 25 μm (1200 g/l) | |
| Gate delay | 2 | μs |
| Gate width | 1.05 | ms |

Raman laser (e.g., CPS532 available
from Thorlabs, Newton, New Jersey)

| | | |
|---|---|---|
| Wavelength | 532 | nm |
| Power | 5 | mW |

| | |
|---|---|
| Spectral width | 0.3 nm FWHM |

Raman detection (e.g., Avaspec-mini available
from Avantes, Apeldoorn, The Netherlands)

| | | |
|---|---|---|
| Raman range | 500-2500 | cm⁻¹ |
| Exposure time | 500 | msec |

| | |
|---|---|
| Optical fiber | 1 mm core diameter |

### EXAMPLES

### Example 1: Sample Preparation

### Polymer Beads

Three different polymer reference samples were prepared in this study. Polystyrene (PS, 441147), Polyethylene (PE, 428043), and Polypropylene (PP, 428116) beads were purchased from Sigma-Aldrich. These beads are reference samples for the purpose of calibration spectra, and identification using fusion data from LIBS and Raman. All beads were put onto slide glass top surface to get both LIBS and Raman spectra in air and room temperature.

### Polymer Powders

Three different polymer powder samples were prepared as the purpose of contamination detection. Polystyrene powder (PS, 9003-53-6, particle size 40 μm) was purchased from Nano Chemazone. Polyethylene (PE, 427772, particle size 30 μm) and Poly Methyl Methacrylate (PMMA, 43982, particle size <20 μm) were purchased from Sigma-Aldrich and Alfa Aesar, respectively. These micro particles are popularly emerged in various food industry such as packaging. For example, polystyrene, either rigid of foamed in disposable cups or food containers etc., can migrate form the packaging into the food. These powders were randomly spread onto top surface of cheese before measurement. In addition, each of 100 mg was pressed for making a tablet pellet for reference spectra.

### Metal Powders

Sulfur powder (414980, particle size >60 μm) was purchased from Sigma-Aldrich since Sulfur is well-known for Raman calibration due to sharp peaks in near spectral range as shown in FIG. 2 (C). In addition, three powder samples were prepared as heavy metal detection. Boron nitride (255475, particle size 1 μm), Zinc (96454), Chromium (266299, particle size 45 μm) were purchased from Sigma-Aldrich. Boron has lower atomic number as 5 and is an element found naturally in leafy green vegetables like spinach, however, it could be also toxic in human body while exposing large amounts. Zinc and Chromium are common contaminants in crops translocated from soil to root tissues and vegetables and bottled water, respectively.

### Pesticide

Roundup, which contains 50% Glyphosate, is most common commercial herbicide in the USA. It is used to kill weeds that compete with crops. Both Raman and LIBS signal detection were performed whether spreading glyphosate liquids on the orange peel using swab surface or not. A clear Raman signal was found when comparing other reference paper, and minor Phosphorus ion emission line in LIBS signal.

### Food Products

Three different kinds of cheese and five different coffee beans were chosen. One Alpine cheese (C6, Charles Arnaud Comte AOP 6 Month Aged) and one Gruyere cheese (C11, Gruyere AOP) were purchased from iGourmet. Another Wisconsin-manufactured cheese (C16) was obtained from local market. Five varieties of coffee bean were purchased from several sources: Italian Dark Roast (OLDE Brooklyn Coffee, Brooklyn, NY; sample C1), Guatemalan Antigua Blend (Copper Moon Coffee, Lafayette, IN; sample C2), Lavazza Super Crema (Luigi Lavazza SpA, New York, NY; sample C3), Despierta tus Sentidos (Nespresso USA Inc., Long Island City, NY; sample C4), Café Cubano Roast (Mayorga Organics, Rockville, MD; sample C5). All LIBS and Raman were conducted towards the back flat side of the coffee bean.

### Example 2: Data Analysis

### Spectrum Acquisition Mode

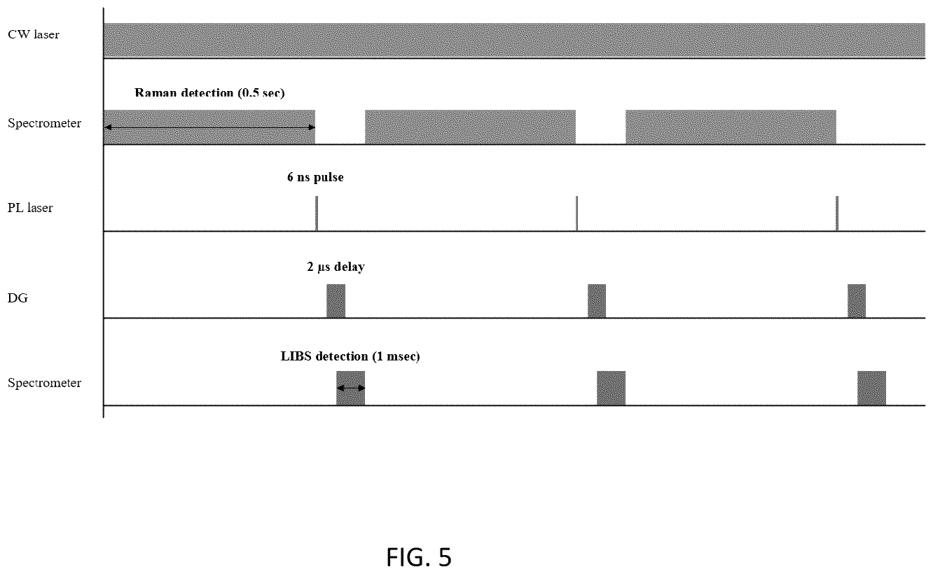

In the developed device, two different data acquisition mode (sequential or simultaneous) could be performed due to sharing same collection optics and spectrometer for both LIBS and Raman. In FIG. 5, Raman signal could be firstly detection within 0.5 second while CW Raman source is continuously working on. After the time period for Raman detection, a pulsed laser was irradiated to detect a pure LIBS signal during 1 msec in same spectrometer. We have demonstrated that there was no overlapped signal within the time period for LIBS detection (1 milli-second). The delay time of 2 μs in delay-generator (DG) was optimized to get a highest signal-to-noise ratio in LIBS.

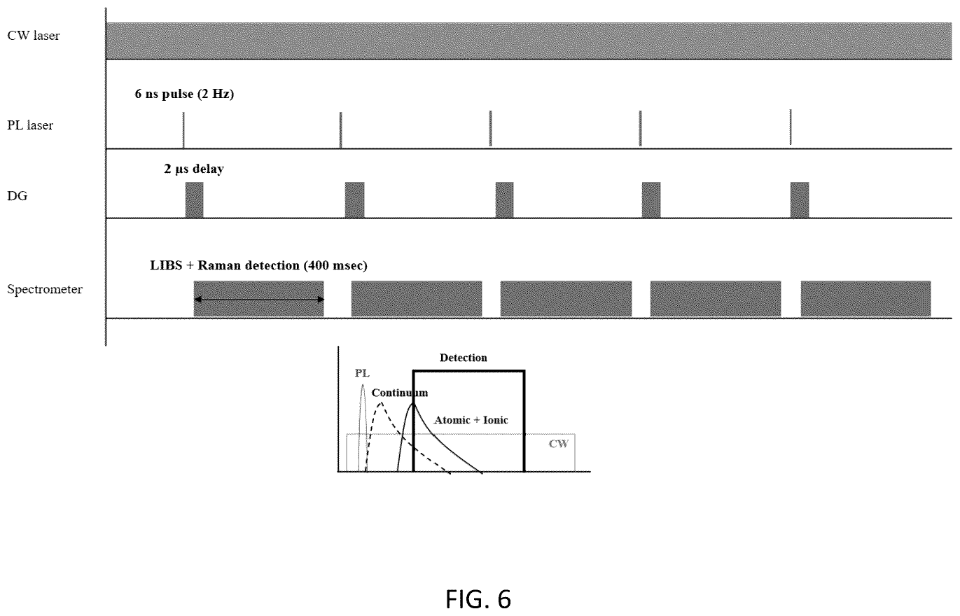

The timeline of simultaneous acquisition was described in FIG. 6. A pulsed laser for LIBS was irradiated with a 2 Hz repetition while a continuous laser for Raman was turning on. After a specific delay from the pulsed laser irradiation, a fixed gate width of 400 milli-second was adjusted to get a simultaneous LIBS and Raman in same spectral range. And second data was acquired from same procedure in above. (Pulsed laser→delay generator→data acquisition →pulsed laser repeatedly)

### Signal Process

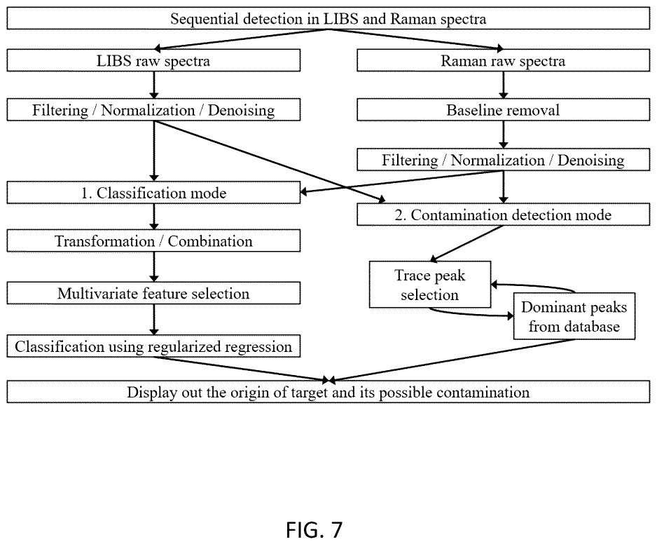

FIG. 7 describes a data processing for both LIBS and Raman. Note that these same procedures such as normalization and baseline removal were conducted in simultaneous detection. In case of raw LIBS and Raman spectra, baseline removal, filtering, total normalization, denoising, and transformation were conducted in all collected data to reduce an uncertainty due to plasma fluctuation. First, trace peak selection was performed to distinguish a possible contaminant. This process was followed from the database, which have dominant peak information of LIBS and Raman for food analysis. Second, classification can be achieved using separate LIBS or Raman signal or combined LIBS and Raman signal after multivariate feature selection before driven a regularized regression. In addition, it was demonstrated that combined LIBS and Raman could be improved a classification accuracy through multivariate feature selection after data fusion. In detail, ANOVA (Analysis of Variance) for univariate analysis and multinomial logistic regression with elastic net regularization (ENET) for multivariate analysis were conducted after data fusion of LIBS and Raman, which same two-dimensional data was performed in each function. To build a classify model, ENET classification could be performed, and SVM (Support vector machine) could be also performed since it was widely used in common spectral analysis. Finally, the classification results or the possible contaminants information will be displayed on the screen.

### Example 3: Results

### Polymer Powders:

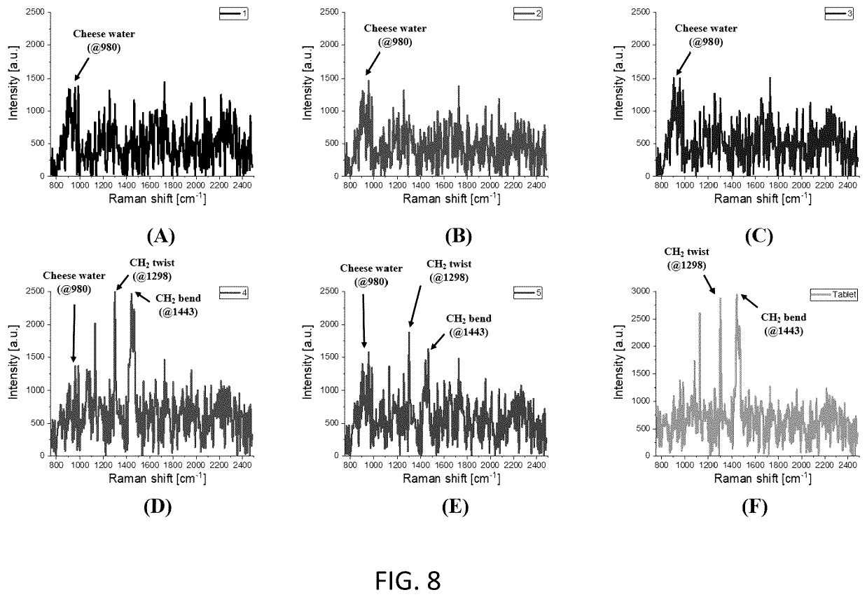

FIG. 8 shows PE powder screening on cheese surface; (A)-(E) as 5 random points and (F) as Reference peak in pellet sample. 50 spectra were accumulated at each point. Points 4 and 5 show similar PE Raman signal when comparing reference signal in (F).

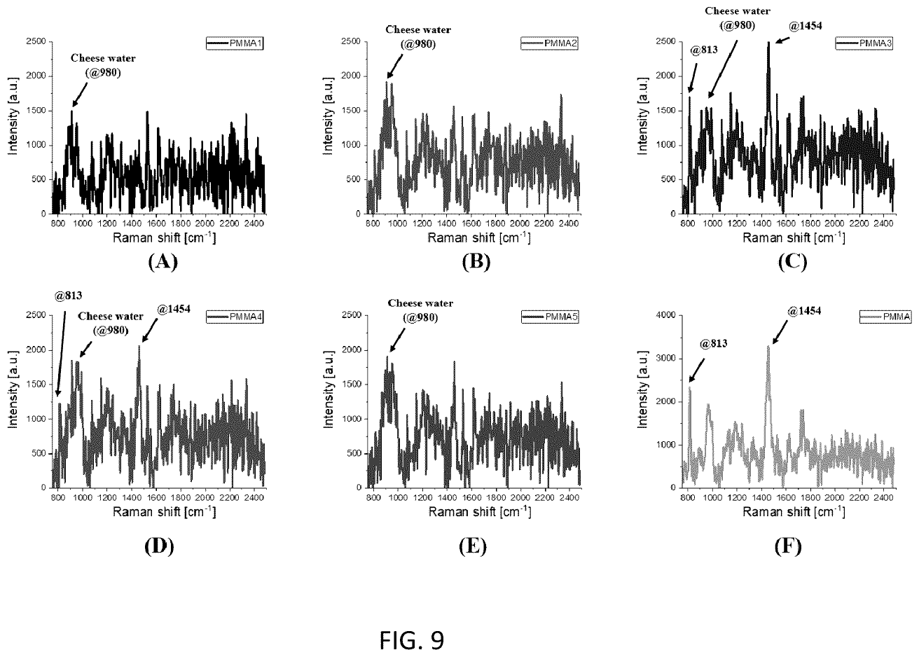

FIG. 9 shows PMMA powder screening on cheese surface; (A)-(E) as 5 random points and (F) as Reference peak in pellet sample. 50 spectra were accumulated at each point. Points 3 and 4 show similar PMMA Raman signal when comparing reference signal in (F).

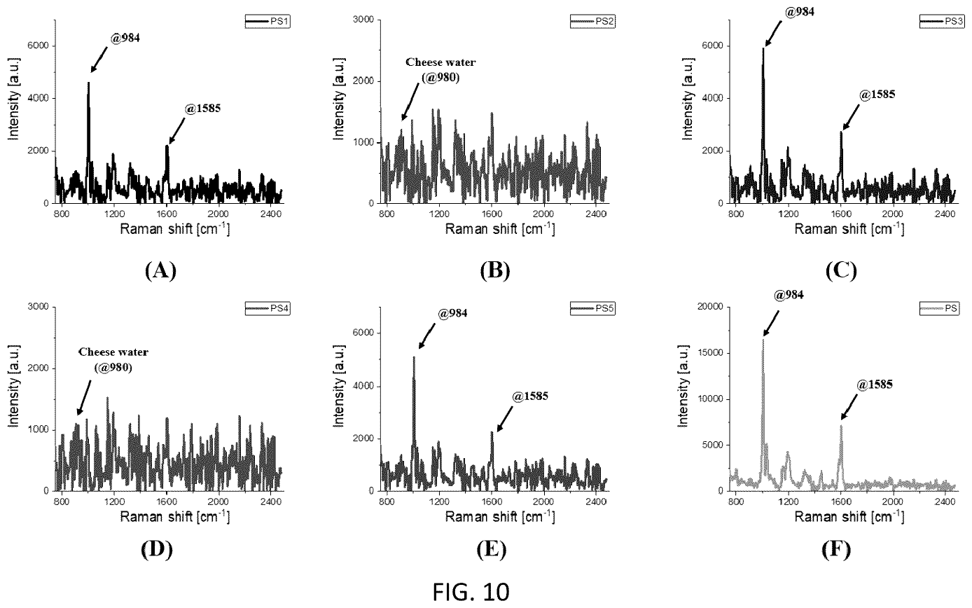

FIG. 10 shows PS powder screening on cheese surface; (A)-(E) as 5 random points and (F) as Reference peak in pellet sample. 50 spectra were accumulated at each point. Points 1, 3, and 5 show similar PS Raman signal when comparing reference signal in (F).

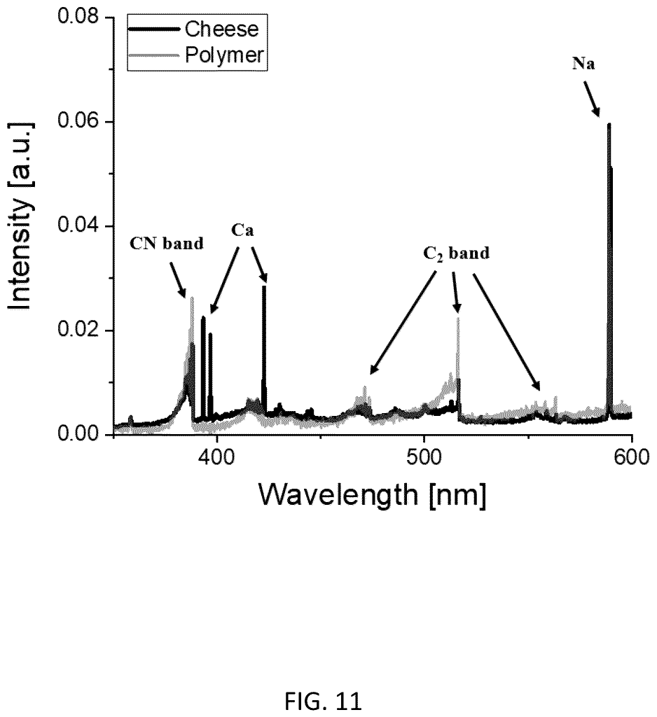

FIG. 11 shows total normalization of 50 averaged LIBS signals in cheese surface and polymer bead. Polymer peaks are overlapped with common food LIBS signals such as molecular peak in LIBS (CN and C₂ band), and elemental peaks (Ca and Na). For micro-plastic detection in food surface, combined LIBS and Raman system is necessary.

### Elements Like Heavy Metals:

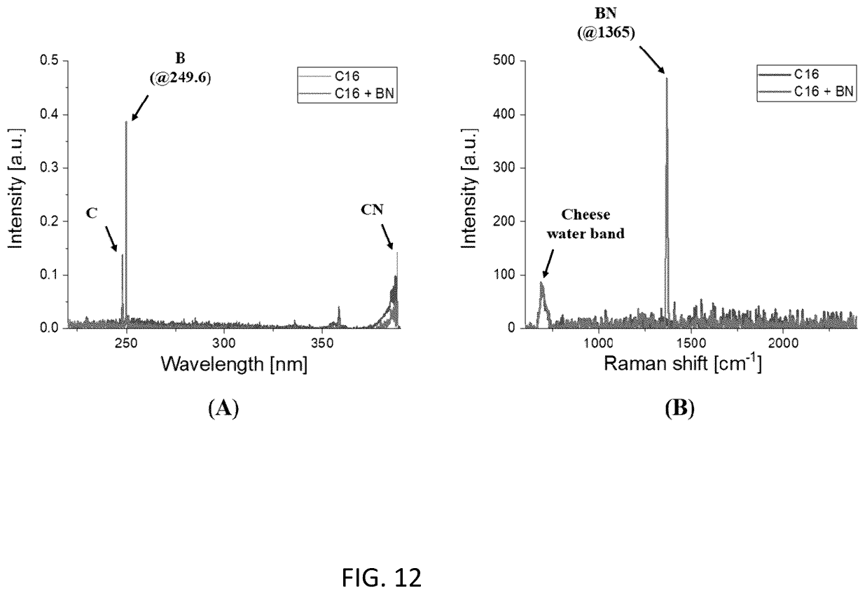

FIG. 12 shows boron powder detection in cheese surface; (A) LIBS data, and (B) Raman data. Total normalization was performed in 50 averaged LIBS spectra in (A). Clear Boron peak is appeared in UV range spectrometer. 50 spectra were averaged in Raman signal after background removal. Clear BN peak was also appeared in Raman data.

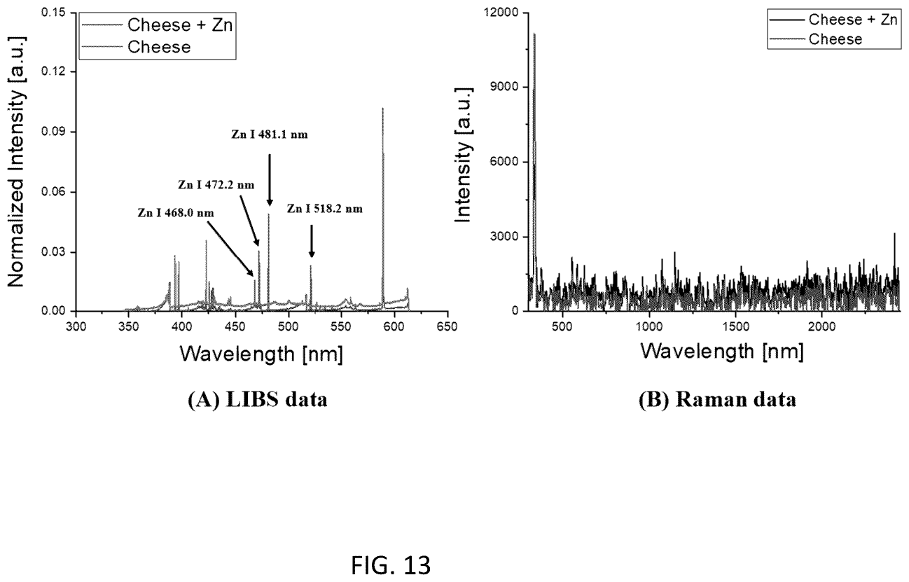

FIG. 13 shows zinc powder detection in cheese surface; (A) LIBS data, and (B) Raman data. Total normalization was performed in 50 averaged LIBS spectra in (A). Clear Zinc peaks are appeared in VIS range spectrometer. 50 spectra were averaged in Raman signal after background removal. However, there were no Zinc peak in Raman data.

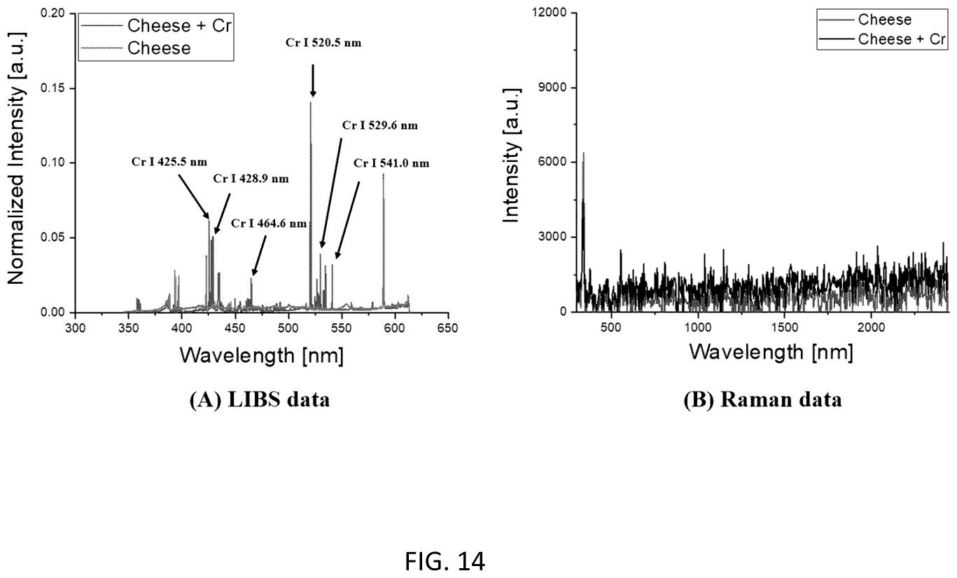

FIG. 14 shows chromium powder detection in cheese surface; (A) LIBS data, and (B) Raman data. Total normalization was performed in 50 averaged LIBS spectra in (A). Clear Chromium peaks are appeared in VIS range spectrometer. 50 spectra were averaged in Raman signal after background removal. However, there were no Chromium peak in Raman data. Note that common cheese signal in LIBS and Raman were appeared together in both two different conditions (cheese only and cheese with powder samples).

### Pesticide

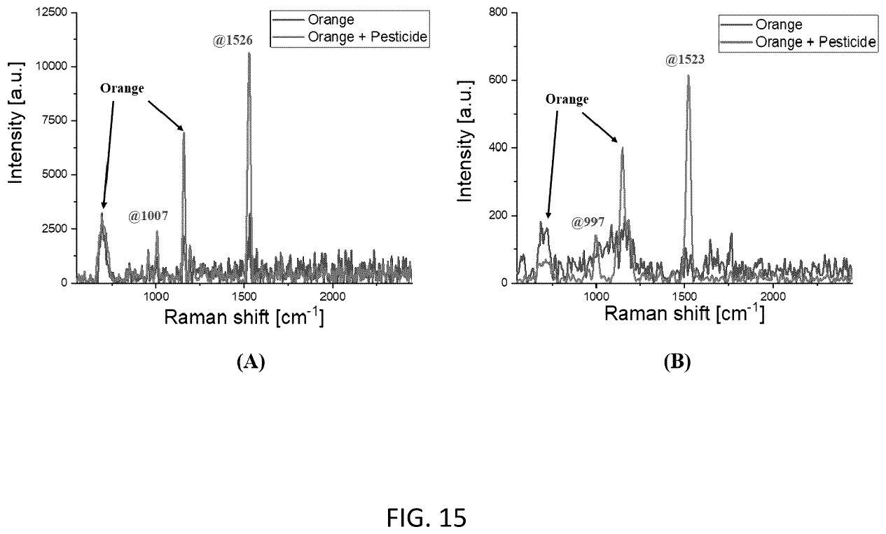

FIG. 15 shows glyphosate Raman data for (A) an exemplary device of the invention (50 accumulated spectra taking 25 sec), and (B) reference device (one single shot data acquired from ICCD taking 1 sec). Only Glyphosate signals were acquired in case of Glyphosate.

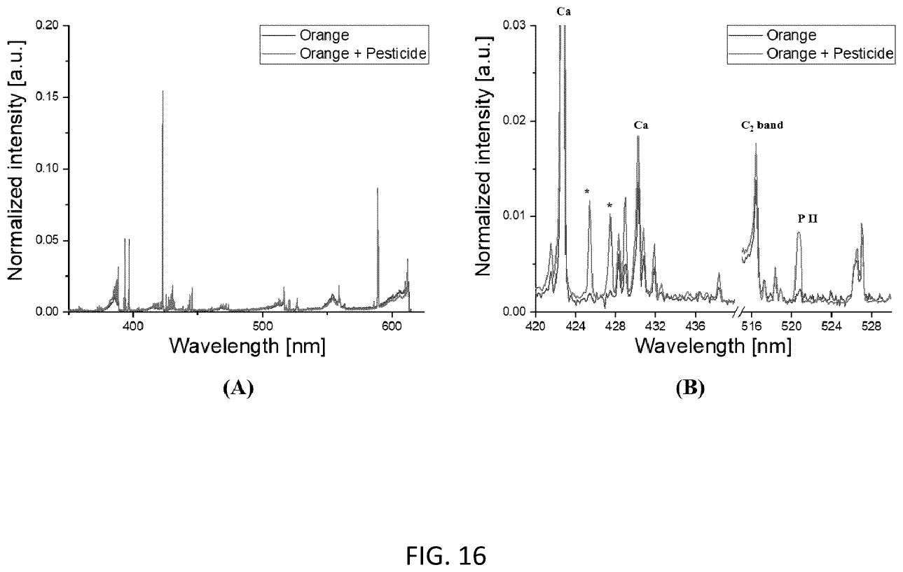

FIG. 16 shows 5 averaged LIBS spectra of Glyphosate after total normalization for (A) full spectral range, and (B) specified spectral range. Only Phosphorous and few unknown minor peaks were acquired in case of Glyphosate spreading on orange peel.

### Adulteration of Food and Polymers

### Cheese

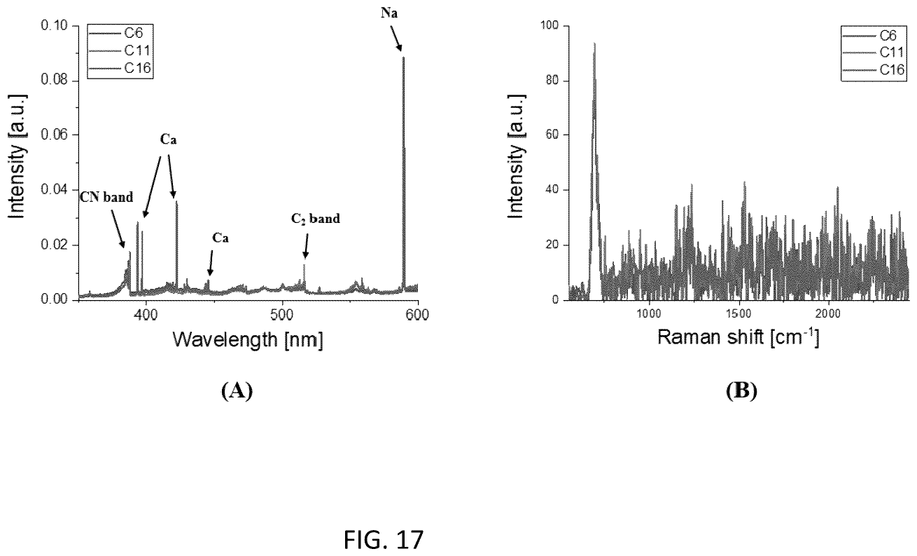

FIG. 17 shows measured data for three different cheeses using (A) LIBS (50 averaged spectra after total normalization), and (B) Raman (50 averaged spectra after background removal).

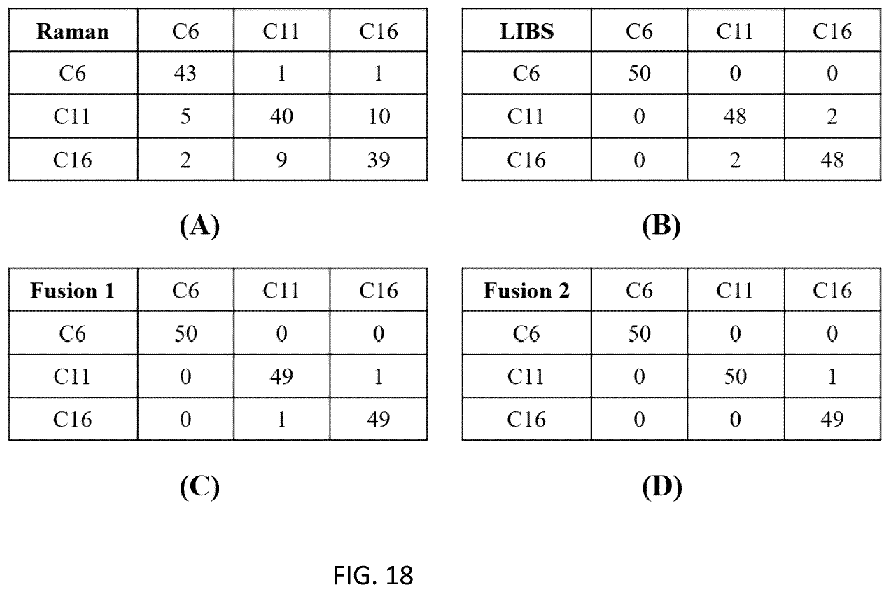

FIG. 18 shows classification of three different cheeses for (A) Raman data only, (B) LIBS data only, (C) combined LIBS and Raman, and (D) combined LIBS and Raman with auto feature selection. All detail processing methods were described above section. Note that in case of processing condition in (D), univariate and multivariate feature selection were performed after combining both LIBS and Raman data might be inducing more effective features for the purpose of classification implying that most overlapped features removed while still existing in the condition of (C).

### Coffee

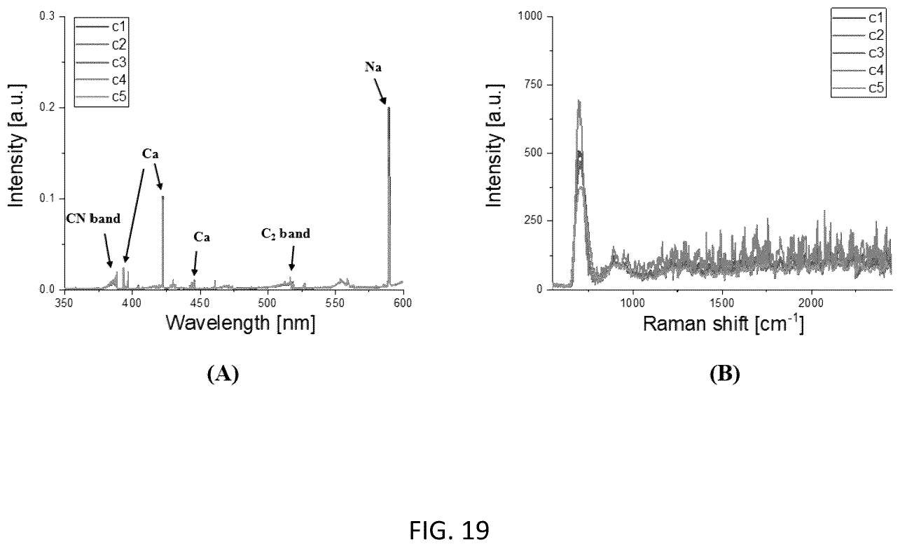

FIG. 19 shows measured data for five different coffee beans for (A) LIBS (50 averaged spectra after total normalization), and (B) Raman (50 averaged spectra after background removal).

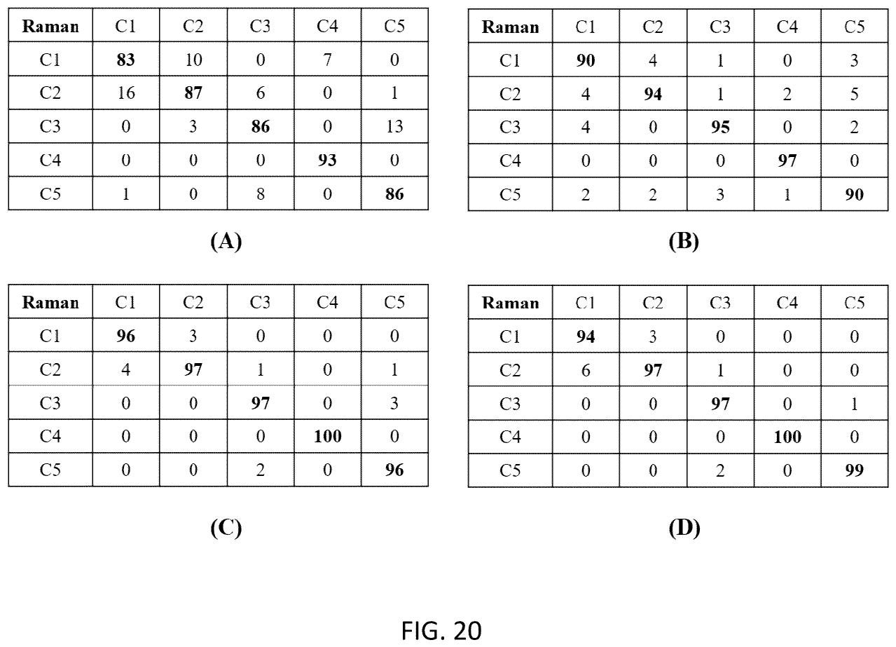

FIG. 20 shows classification of five different coffee beans for (A) Raman data only, (B) LIBS data only, (C) combined LIBS and Raman, and (D) combined LIBS and Raman with auto feature selection.

### Polymer

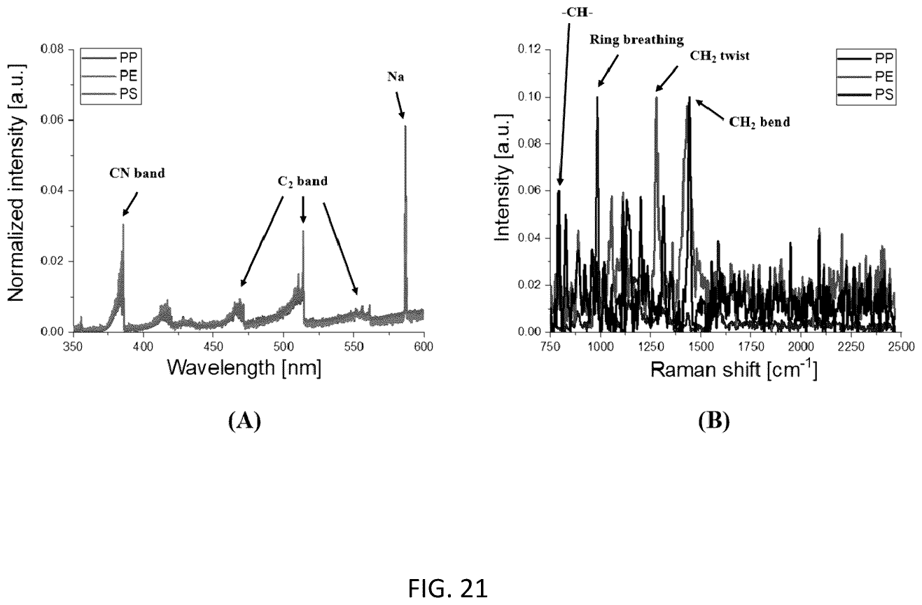

FIG. 21 shows measured data for three different polymer beads for (A) LIBS (50 averaged spectra after total normalization), and (B) Raman (50 averaged spectra after background removal).

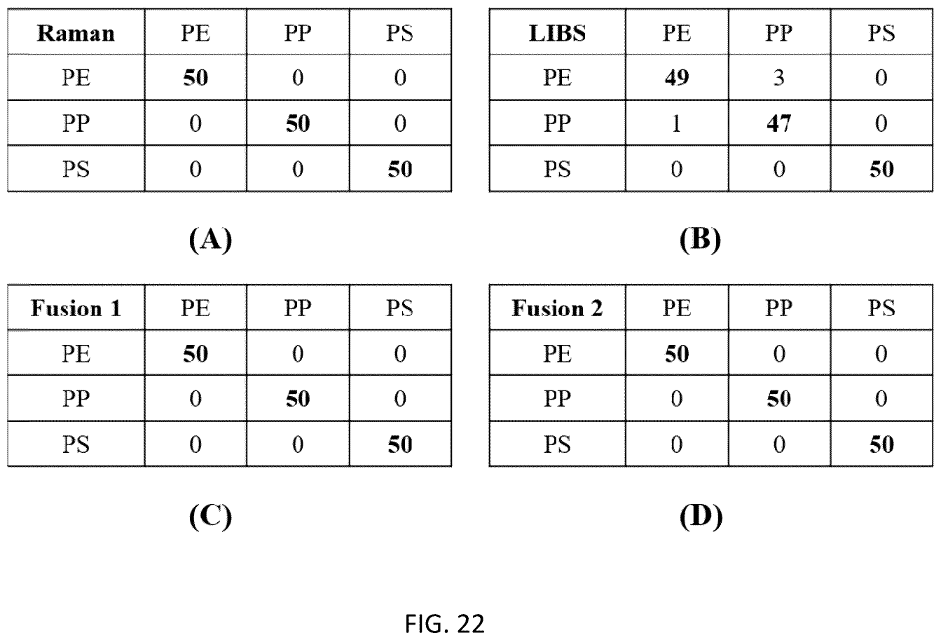

FIG. 22 shows classification of three different polymer beads for (A) Raman data only, (B) LIBS data only, (C) combined LIBS and Raman, and (D) combined LIBS and Raman with auto feature selection.

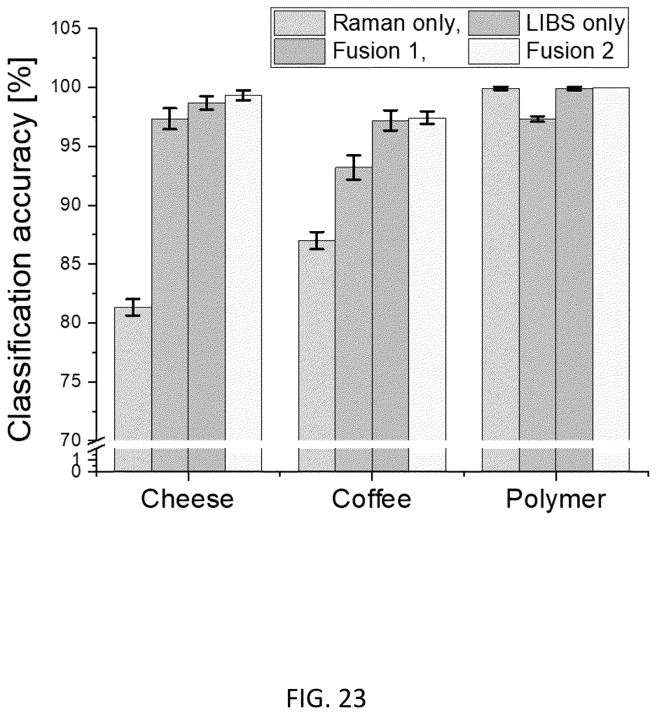

FIG. 23 shows classification summary plots for (A) Cheeses, (B) Coffee beans, and (C) Polymer beads with 10 running circles.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

1. A system comprising:
a first excitation source applying a first excitation energy to a point on a sample stage;
a second excitation source applying a second excitation energy to the point simultaneously to the first excitation energy; and
a single spectrometer positioned to receive and independently process energy emitted from the point in response to both the first and second excitation energies.

2. The system of claim 1, wherein the first source is a pulsed excitation source.

3. The system of claim 2, wherein the first source is a laser-induced breakdown spectroscopy (LIBS) source.

4. The system of claim 3, wherein the first source is a near-infrared region wavelength laser.

5. The system of claim 4, wherein the first source is a laser having a wavelength of about 1064 nm, energy of about 10 mJ, and about a 0.2 nm FWHM beam width, and applied in a pulse of about 6 ns.

6. The system of claim 2, wherein the second source is a continuous excitation source.

7. The system of claim 6, wherein the second source is Raman source.

8. The system of claim 7, wherein the second source is a visible laser.

9. The system of claim 8, wherein the second source is a laser having a wavelength of about 532 nm, energy of about 5 mW, and about a 0.3 nm FWHM beam width.

10. The system of claim 1, further comprising one or more mirrors and one or more lenses to direct and focus the first excitation energy and the second excitation energy on the point on the sample stage.

11. The system of claim 10, wherein the one or more mirrors comprise a dichroic mirror operable to reflect excitation energy from the second excitation source and allow excitation energy from the first excitation source to pass therethrough.

12. The system of claim 10, wherein the one or more lenses comprise a focusing lens.

13. The system of claim 1, further comprising collection optics associated with the single spectrometer and one or more collecting lenses positioned between the point and the collection optics.

14. The system of claim 13, further comprising a notch filter positioned between two collecting lenses between the point and the collection optics.

15. The system of claim 14, wherein the notch filter is tuned to filter out a wavelength of the second source.

16. The system of claim 13, wherein the collection optics comprise an optical fiber coupled to the single spectrometer.

17. The system of claim 1, wherein the single spectrometer is a visible range spectrometer

18. The system of claim 1, wherein the system is contained in a container having a total volume of about 750 cm3 or less.

19. The system of claim 1, wherein the system is contained in a container having a longest linear dimension of about 15 cm.

20. The system of claim 1, wherein the first source is a pulsed excitation source and the second source is a continuous excitation source, the system further comprising a processor coupled to a tangible non-transient memory operable to acquire data from the single spectrometer for the second source in between pulsing of the first source.

21. The system of claim 1, wherein the sample stage is moveable in three axes.

22-39. (canceled)
